(19) United States
(12) Patent Application Publication
(10) Pub. No.: US 2005/0032033 A1
(43) Pub. Date: Feb. 10, 2005

(54) Identifying, monitoring, and sorting genetically modified plant portions

(76) Inventors: Denny C. Winterboer, Milford, IA (US); Katie A. Thompson, Hartley, IA (US)

Correspondence Address: JONES DAY, 222 EAST 41ST ST, NEW YORK, NY, 10017, US

(21) Appl. No.: 10/872,169

(22) Filed: Jun. 18, 2004

Related U.S. Application Data
(60) Provisional application No. 60/479,923, filed on Jun. 19, 2003.

(51) Int. Cl. C12Q 1/00; C12Q 1/34; C12Q 1/66

(52) U.S. Cl. 435/4; 435/18; 435/8

(57) ABSTRACT

The present invention relates to compositions and methods for identifying, monitoring, and sorting specific genetically-modified plant portions from other genetically-modified plant portions. The present invention also relates to compositions and methods for identifying, monitoring, and sorting specific genetically-modified plant portions from non-genetically modified plant portions where both are present in a mixture. Either or both of the genetically modified plant portions or the non-genetically modified plant portions can comprise a distinguishable marker which is identified and used for sorting such mixtures of plant portions. The present invention is also directed toward kits useful in the methods disclosed herein. The compositions, methods, and kits of the present invention are used inter alia in high-throughput, sorting systems for identity preservation of a seed stock, to provide seed populations that are free of genetically-modified seeds, to isolate hybrid seed uncontaminated with selfed seed, and to isolate one type of genetically-modified plant portion from a mixture of genetically-modified plant portions.

### 1. FIELD OF THE INVENTION

The present invention relates to compositions and methods for identifying, monitoring, and/or sorting plant portions of a first plant from plant portions of a second plant that are present in a mixture, wherein either, both, or neither of the first and the second plant is a genetically-modified plant. Either or both of the plant portions of the first and the second plant can comprise a distinguishable marker which is identified and used for sorting such mixtures of plant portions.

The methods of the present invention are used inter alia in high-throughput, automated sorting systems for identity preservation of a seed stock, to provide seed populations that are free of genetically-modified seeds, and to isolate hybrid seed uncontaminated with selfed seed.

### 2. BACKGROUND OF THE INVENTION

Genetic engineering or genetic modification of plants provides benefits (improved nutritive value, herbicide resistance, production of edible vaccines and other therapeutic products) and presents potential risks as well (introduction of a known allergen/epitope into a plant where that allergen is not normally found; introduction of a previously unidentified epitope into a new food—e.g. a previously-unidentified allergen from Brazil nuts was inadvertently introduced into food plants).

Consumer preferences and fears are leading to Government regulation and requirements for warning labels on food. Consequently, a market demand has been created for grain and plant products that are certifiably-free of genetically modified plant materials.

In addition, the development of high-value genetically-modified plant products has led to the need to create pure or enriched populations of the desired plant product from a starting mixture of plant products that can include unmodified or other, unrelated genetically-modified plant products.

There are inherent problems in providing identity-preserved products, including the existence of contaminated seed stocks resulting from cross pollination of seed crops, which can result from wind-borne pollen or pollen carried by bees etc. Uncontrolled cross pollination may also lead to liability damages where genetically-modified pollen contaminates a non-genetically-modified crop of another.

In addition cross pollination of growing crops as well as “mechanical” cross-contamination of genetically-modified and non-genetically-modified crops and grains can occur during planting (augers) harvesting (combines, grain carts etc), transport (trucks, rail cars) and storage (grain elevators).

Current methods and approaches to avoid contamination by genetically-modified-plants and to maintain varietal purity (or “identity preservation”) include the creation of buffer zones surrounding crops of genetically-modified-plants and the genetic modification of the genetically-modified plant to establish conditional lethality etc. Other such methods include designing and operating crop production and handling facilities in a manner intended to ensure total physical separation of varieties during all stages of production and distribution.

Hybrid plants grown from hybrid seed frequently display desirable traits that reflect the heterotic effects obtained by crossing two genetically distinct plant lines. The progeny of such hybrid seeds often display agronomic characteristics that are superior to both parent strains. Accordingly, seed stocks that are certifiably hybrid provide better-performing crops, as compared to those developed from open-pollinated seed, and therefore have economic value. However, production of hybrid seed stocks free of self-pollinated seed is a technical challenge that has been approached using mechanical, chemical, genetic and recombinant methods such as those described, for example, in U.S. Pat. No. 6,184,439 B 1, which is incorporated herein by reference in its entirety. Accordingly, there is a need for methods that can be used to distinguish, separate, and certify hybrid seed, derived from two defined parent plants, from self-pollinated seed.

As noted above, there is a demand for certification of crops, and products derived therefrom, as free of genetically-modified, differently-genetically-modified, or other undesirable plants or portions thereof. There are methods for determining whether a given sample is a mixture of genetically-modified-material and non-genetically-modified material including: ELISA, bioassays (e.g. seed germination and or plant growth in the presence of a selective agent), and PCR analyses. However these methods are not only destructive, they are not amenable to sorting processes; that is, these methods are useful for detecting but not for sorting, enriching, or purifying mixed stocks of plants or plant portions.

There is a need for a non-destructive method for monitoring, identifying and/or sorting: (a) genetically-modified from non-genetically-modified plant portions; (b) different genetically-modified plant portions form one another; and (c) different non-genetically-modified plant portions form one another, where the plant portions can be, but are not limited to, seeds.

### 3. SUMMARY OF THE INVENTION

The present invention is directed toward compositions and methods for detecting plants or portions thereof that comprise a distinguishable marker, in which the plant or plant portion is contacted with an agent that interacts with the marker to provide a detectable signal. The plant or plant portions useful in the methods of the present invention include intact plants, roots, tubers, berries, rhizomes, stems, leaves, flowers, shoots, seeds, fruits, grains, and seeds. In certain embodiments, the plant portion is a seed.

The invention is further directed toward methods for monitoring and/or sorting mixtures of plants or portions of plants, where only some members of the mixture comprise the distinguishable marker. In certain aspects of this embodiment, the mixture comprises a plurality of genetically-modified plant portions and/or a plurality of non-genetically-modified plant portions, wherein one or more of the genetically-modified plant portions and/or non-genetically-modified plant portions comprises a distinguishable marker. The marker is identified and used to identify, monitor, and/or sort one or more of the genetically-modified plant portions and/or non-genetically-modified plant portions present in the mixture. In another aspect of this embodiment, the mixture of plant portions comprises a plurality of non-genetically modified plant portions in which at least one of the non-genetically modified plant portions comprises a distinguishable marker.

In certain embodiments, the marker is detected by contacting the mixture with an agent that interacts with the marker to provide a detectable signal, thereby identifying a plant or plant portion, which is then monitored in and/or separated from the mixture. In certain embodiments, the identification of the plant or plant portion comprising the distinguishable marker, and the monitoring and separation of the identified plant or plant portion are performed using commercially available, automated monitoring and sorting equipment. In certain embodiments, the plant portion is a seed, and the equipment is automated.

In another embodiment, the present invention is directed toward identification, monitoring, and separating plant portions in which the distinguishable marker is a detectable marker and is identified in the absence of an exogenously-provided agent, such as a detection agent.

In a particular embodiment, the present invention is directed toward a method for sorting a mixture comprising one or more types of genetically modified seeds that carry one or more markers, and therefore are distinguishable from non-genetically modified seeds. In one embodiment, the distinguishable marker is an enzyme. In this method, the seed mixture is contacted with a detection agent that comprises a substrate that is chemically altered by the enzyme; i.e. the substrate may be cleaved (e.g. hydrolyzed) or otherwise modified by the enzyme. The mixture is contacted under appropriate conditions and for a suitable period of time for the enzyme to cleave or otherwise modify a sufficient amount of the substrate to provide a detectable signal that is sufficient to distinguish one component of the mixture from another. The substrate used is one that, upon cleavage or other modification, yields at least one detectable product, such as a chromophoric, fluorescent, or chemiluminescent cleavage product that remains associated with the genetically-modified seed in which the distinguishable marker is expressed, thereby labeling such seeds. Such labeled seeds are identified by the presence of the label using manual or automated detection means and separated from the mixture using manual or automated separation means. In one aspect of this embodiment, genetically modified, labeled seeds are separated from the mixture and collected separately, while in another aspect, non-genetically modified, non-labeled seeds are separated from the mixture and collected separately. In another aspect, one type of genetically-modified seed is separated from another, differentially-modified, genetically-modified seed. In another aspect of this embodiment, the mixture comprises a plurality of non-genetically-modified seeds, wherein at least one member of the plurality of non-genetically-modified seeds comprises a distinguishable marker.

In a further embodiment, chemical modification of a detection agent leads to a detectable product such as, but not limited to, visible light. In this embodiment, contacting of the mixture of plant portions with the detection agent and identification of plant portions elaborating light are closely spaced temporally to provide efficient and accurate identification, monitoring, and separation of plant portions exhibiting light production.

In another embodiment, the compositions and/or methods of the present invention are used for sorting a mixture of seeds that comprises genetically-modified seeds as well as non-genetically-modified seeds, in which the non-genetically-modified seeds comprise a distinguishable marker, which is an enzyme. In this aspect of the invention, non-genetically-modified seeds are positively identified using manual or automated detection means and actively separated from a mixture of seeds that comprises genetically-modified seeds, using manual or automated separation means. In this method, the seed mixture is contacted with a detection agent that comprises a substrate that is cleaved or otherwise modified by the marker enzyme. The mixture is contacted under suitable conditions and for a suitable period of time for the enzyme to cleave or otherwise modify a sufficient amount of the substrate to provide a detectable signal. The substrate used is one that, upon cleavage or other modification, yields at least one detectable product, such as a chromophoric, fluorescent, or chemiluminescent cleavage product that remains associated with the non-genetically-modified seed in which the distinguishable marker is expressed, thereby labeling the non-genetically-modified seeds. Such labeled seeds are identified by the presence of the label using automated detection means and then separated from the mixture using manual or automated separation means. In one aspect of this embodiment, non-genetically modified, labeled seeds are separated from the mixture and collected separately, while in another aspect, genetically modified, non-labeled seeds are separated from the mixture and collected separately.

In certain embodiments of the present invention, a plant portion mixture is contacted with a composition comprising a detection agent, which is cleavable or otherwise modifiable by an enzymatic activity present in, e.g., both genetically-modified and non-genetically modified plant portions present in the mixture, and a second molecule. In this embodiment, the second molecule is a selective inhibitor of the enzymatic activity present in either the genetically-modified or in the non-genetically modified plant portions present in the mixture, but not both. Accordingly, the enzymatic activity that is resistant to the selective inhibitor serves as a distinguishable marker, in the presence of that selective inhibitor.

The present invention is also directed toward methods for purifying a hybrid seed population in the presence of seed arising from self-fertilized plants (“selfed” seed) where each hybrid seed parent comprises a marker not present in the other parent. In this embodiment, those seeds comprising both parental markers are sorted from a seed population comprising selfed seeds as well as the desired hybrid seeds, by identifying, using manual or automated detection means, and separating, using manual or automated separation means, only those seeds comprising both parental markers. In one aspect of this embodiment, the sorting is carried out sequentially, whereby plant portions displaying a first detectable marker are collected and those collected plant portions are then sorted a second time to collect those plant portions displaying the second detectable marker as well.

In certain embodiments of the methods of the present invention, the contacting is automated.

In further embodiments, the contacting is carried out by applying detection agent at the time the plants are first planted. This is done by planting with equipment including, in one non-limiting example, a Case IH, 12-row 30-inch planter (Case IH, New Moline, Ill.) and planting seeds, such as but not limited to soybean seeds, in 30-inch rows. The seeds are contacted by passing the detection agent through a pump attached to the planter (i.e. “in furrow” application) that delivers a liquid suspension of detection agent into the furrow created by the planting equipment and next to the seeds deposited by the planting equipment. In one non-limiting aspect of this embodiment, the detection agent is carried in saddle tanks on the planter. In another embodiment, the seeds are contacted by mixing a formulation of the detection agent with the seed before planting (“pre-treated seed” or “seed treatment”).

In further embodiments, the contacting is performed by applying detection agent while the plant portions are in a growth or maturation phase on live plants, by spraying using equipment such as, but not limited to, a 90-foot RoGator sprayer (AgChem, Inc., Jackson, Minn.) that sprays detection agent at a rate such as, in one non-limiting example, 24 fluid ounces of solution per acre. In another aspect, the contacting is performed on live plants by irrigation while the plant portions are in a growth or maturation phase.

In still further embodiments, the contacting is performed by spraying harvested plant products as they are transferred to or from storage or shipping facilities (“binside application”). In one non-limiting example, the application device includes a FAST (Farmer-Applied Seed Treater) liquid sprayer from TraceChem, Inc. (Perkin, Ill.) capable of spraying 60 ounces per minute of a liquid composition comprising the detection agent onto plant portions. The plant portions may be transferred on a device such as but not limited to a Sudenga 65-foot Auger (Sudenga, George, Iowa) capable of moving plant portions, such as but not limited to soybean seed, at a rate of 3,000 pounds per minute. In one embodiment, the FAST sprayer nozzle is at the intake of the auger and the seed conveyed up the auger to the outlet before being deposited into the storage facility.

In preferred embodiments, the reaction between the target plant portion and the detection agent will occur at ambient temperatures as found during growth, maturation, harvest, storage, shipping, or processing.

In certain embodiments the marker is detected without the need for contacting with an exogenous detection agent. In one aspect of this embodiment, the marker is a fluorescent protein, such as, but not limited to the green fluorescent protein of Aequorea victoria, or a derivative thereof with enhanced fluorescence in plant tissue. In another aspect of this embodiment, the marker is firefly luciferase, or a bacterial luciferase such as but not limited to bacterial luciferase expressed by the lux genes of Vibrio fischeri and Vibrio harveyi.

In another embodiment, the present invention is directed toward a genetically-engineered or genetically-modified plant or plant portion that expresses only the luxA and luxB gene products of Vibrio fischeri. In this instance, a detection agent, n-decanal, is applied to provide identification of genetically-modified plants or portions thereof expressing luxA and luxB gene products.

In another embodiment of the present invention, the marker comprises all or a portion of a biosynthetic pathway that provides a detectable signal. In certain aspects of this embodiment, the detectable product is an intermediate, shunt product, or the final product of the biosynthetic pathway or portion thereof. In another aspect of this embodiment, more than one plant portion in a mixture of plant portions comprises the biosynthetic pathway or portion thereof, but the amount and/or tissue-specific accumulation of the detectable product is sufficiently different between plant portions to permit the efficient and accurate identification, monitoring, and separation of one plant portion from another.

In further embodiments, the reaction can occur at specific, regulated temperatures found in a storage bin, such as but not limited to a 10,000 bushel Butler Bin (Butler Mfg., Kansas City, Mo.) that will maintain a 50° F. core temperature.

The detection can also occur at various stages of production and handling, including but not limited to, processing at harvest, handling at cooperative storage facilities (“elevators”), loading at shipping terminals, or preparation at food processing facilities. The detection can be incorporated into testing currently performed at delivery points, including, e.g., tests for moisture, foreign material (“FM”), protein, and oil, according to methods well known to those skilled in the art.

In preferred embodiments, the methods of the present invention are “non-destructive,” i.e. they do not substantially disrupt the plant portion, and the methods are “non-lethal,” i.e., they do not substantially decrease the viability of the treated seeds or other plant portion, such as but not limited to tubers. As used herein, the phrase “does not substantially reduce the viability” means that a population of seeds or other plant portion treated according to the methods disclosed herein, retains, in certain embodiments at least 75% viability, preferably at least 85%, more preferably at least 90%, even more preferably at least 95%, and, most preferably, at least 97% viability. Viability is measured by germination testing of a statistically meaningful number of treated and untreated seeds, using standard methods appropriate for each cultivar, generally according to methods well known in the art (Practical Statistics and Experimental Design for Plant and Crop Science, Alan G. Clewer and David Scarisbrick, John Wiley and Sons, March 2001).

In other embodiments, the methods and compositions of the present invention are “lethal,” i.e. they do substantially reduce the viability of the treated seeds or plant portions. In yet another embodiment, the methods are lethal only to specific, genetically-modified plant portions in a mixture.

In a preferred embodiment, the methods and compositions of the present invention are “non-toxic” ; i.e. they are acceptable treatments of plant portions that are or will become food products for human consumption. In other embodiments, the methods and compositions of the present invention are toxic and plant portions thus treated are not suitable for human consumption but may be suitable for animal food or other industrial uses such as chemical extraction.

The methods of the present invention are used for manual and automated identification and separation of genetically-modified plant portions and/or genetically non-modified plant portions selected from the group consisting of, but not limited to, corn, soybean, oat, rye, sunflower, wheat, rice, barley, beet, canola, cotton, potato, chicory, tomato, carnation, melon, tobacco, pea, coffee, and mustard plant seeds, as well as mixtures thereof.

In yet another embodiment of the present invention, the marker is an enzyme selected from, but not limited to, the group of enzymes consisting of β-D-glucuronidase, acetolactate synthase, dihydroflavonol reductase, flavonoid 3p 5p hydroxylase, neomycin phosphotransferase II, nopaline synthase, β-lactamase, phosphonothricin N-acetyltransferase, 5-enolpyruvylshikimate-3-phosphate synthase, glyphosate-resistant 5-enolpyruvylshikimate-3-phosphate synthase, glyphosate oxidoreductase, barnase ribonuclease, acetyl CoA carboxylase, DNA adenine methyl transferase, S-adenosylmethionine hydrolase, aminocyclopropane cyclase synthase, thioesterase, helicase, bromoxynil nitrilase, replicase (RNA-dependent RNA polymerase), and Δ-1, 2 desaturase.

In still further embodiments of the present invention, the marker is an enzyme selected from, but not limited to, the group of enzymes consisting of 3 keto thiolase, 3-hydroxy-3-methylglutaryl CoenzymeA reductase, 3 hydroxyl trichoecene acetyltransferase, 4 coumarate:CoA ligase, ACC deaminase, ACC synthase, aceto acetylCoA reductase, acetohydroxyacid synthase variant, acetolactate synthase, acetyl CoA carboxylase, ACP acyl ACP thioesterase, ACP thioesterase, acyl ACP desaturase, acyl CoA reductase, acylACP thioesterase, adenine methylase, ADP glucose pyrophosphorylase, alpha amylase, amino glycoside adenyl transferase, amino polyol amine oxidase, aminoglycoside 3′ adenylyltransferase, aminoglycoside acetyltransferase, amylase, anionic peroxidase, apotyrosinase, ascorbate peroxidase, asparagine synthetase, aspartokinase, aspartokinase II, homoserine dehydrogenase, β-glucuronidase, β-keto acyl Coenzyme A synthase, bamase, beta glucanase, betaine aldehyde dehydrogenase, branching enzyme (TB 1), caffeate O-methyltransferase, campesterol synthesis (DIM) gene, cellulase, chitinase, chitobiosidase, chloramphenicol acetyltransferase, choline oxidase, cinnamate 4 hydroxylase, cinnamyl alcohol dehydrogenase, citrate lyase, citrate synthase, cyanamide hydratase, cyclin dependent kinase, cyclodextrin glycosyltransferase, cystathionine beta lyase, cystathionine synthase, dehydroascorbate reductase, delta 12 saturase, delta 9 desaturase, deltal2 desaturase, deltal5 desaturase, diacylglycerol acetyl transferase, dihydrodipicolinate synthase, dehydroflavonol reductase, dihydrofolate reductase, dihydropteroate synthase, divinyl ether synthase DNA adenine methylase, DNA methyltransferase, double stranded ribonuclease, elongase, endoxyloglucan transferase, EPSPS, esterase, ethylene forming enzyme, exochitinase, fatty acid elongase, flavin amine oxidase, flavonol 3 hydroxylase, formamido pyrimidine DNA glycosylase, fructosyl transferase, galactanase, galactinol synthase, glucanase, glucose oxidase, glutamate dehydrogenase, glutamine synthetase, glutathione reductase, glutathionine transferase, glycerol 3 phosphate acetyl transferase, glyphosate oxidoreductase, helicase, hemicellulase, histone deacetylase, homoserine dehydrogenase, hydroperoxide lyase, hygromycin phosphotransferase, hyoscamine 6-β-hydroxylase, IAA monooxygenase, inositol hexaphosphate phosphohydrolyase, inositol methyl transferase, invertase, isoamylasetype starch debranching enzyme, isoflavone synthase, isopentenyl transferase, ketoacylACP synthase, laccase, lacZ, levansucrase, L-gulono-gamma-lactone oxidase, lignan biosynthesis protein, lignin peroxidase, lipoxygenase, luciferase, lysine ketoglutarate reductase, lysine2 gene, lysophosphatidic acid acetyl transferase, lysophosphatidyl choline acetyl transferase, lysozyme, mercuric ion reductase, monooxygenase, N-acetyl glucosidase, nitrilase, nopaline synthase, NptII, O-acyl transferase, oleayl ACP thioesterase, omega 3 desaturase, omega 6 desaturase, O-methyltransferase, oxalate oxidase, palmitoyl thioesterase, parathion hydrolase, pectate lyase, pectin esterase, pectin methylesterase, pentenlypyrophosphate isomerase, peroxidase, phosphinothricin acetyl transferase, phosphoglucomutase, phytoene destaurase, phytoene synthase, pinoresinol lariciresinol reductase, pinoresinol reductase, polygalacturonase, polyhydroxybutyrate synthase, polyphenol oxidase, protease, protein kinase, putrescine N-methyltransferase, pyruvate decarboxylase, quinolinate phosphoribosyl transferase, receptor kinase, receptor kinase (Xa21 resistance gene), recombinase, reductase, replicase, resveratrol synthase, ribonuclease, S-adenosylmethione decarboxylase, S-adenosylmethionine transferase, saccharopine dehydrogenase, S-adenosylmethione hydrolase, salicylate hydroxylase, secoisolariciresinol dehydrogenase, serine threonine protein kinase, sorbitol 6 phosphodehydrogenase, sorbitol dehydrogenase, sorbitol synthase, starch branching enzyme II, starch debranching enzyme, starch synthase, stilbene synthase, streptomycin aminoglycoside adenyl transferase, streptomycin phosphotransferase, sucrose nonfermenting-related protein kinase, sucrose phosphate synthase, sucrose synthase, superoxide dismutase, thiamine biosynthetic enzyme, thioesterase, thiolase, threonine deaminase, trans aldolase, trehalase, trichodiene synthase, tryptophan monooxygenase, tyrosinase, UDP glucose glucosyltransferase, UDP glucose 4′epimerase, and violaxanthin de-epoxidase.

In certain embodiments of the methods of the present invention, the genetically-modified plants, plant portions, or, in preferred embodiments, seeds comprise a transgene, and in one aspect of these embodiments, the transgene encodes the distinguishable marker.

In certain embodiments of the present invention, the mixture of plants or plant portions to be sorted includes genetically engineered or genetically modified plants or plant portions that comprise the distinguishing marker. In this embodiment, using automated equipment as described above, the genetically modified plant or plant portion is labeled, identified, detected, and sorted (i.e. “ejected”) from the automatically conveyed mixture. In preferred embodiments, the sorted, non-genetically engineered or non-genetically modified seed contains less than about 10% genetically modified or genetically engineered plants or plant portions, in more preferred embodiments, less than about 5% genetically modified or genetically engineered plants or plant portions, less than about 2% genetically modified or genetically engineered plants or plant portions, and most preferably, less than 1% genetically modified or genetically engineered plants or plant portions. In other embodiments, purified, non-genetically engineered or non-genetically modified plants or plant portions are subjected to further cycles of purification according to the present invention to provide non-genetically-engineered or non-genetically modified plants or plant portions comprising 0.1% or less of genetically modified or genetically engineered plants or plant portions. In one aspect of this embodiment, the plant portions are seeds.

The present invention is also directed to a composition useful in a method for detecting and/or separating a plant portion of a first plant from a plant portion of a second plant in a mixture thereof, wherein plant portions of the first plant comprise a distinguishable marker, which marker is an enzyme. Such compositions comprise a detection agent and at least one compound selected from the group consisting of a surfactant and a selective inhibitor, and combinations thereof. The selective inhibitor does not substantially inhibit the marker enzyme present in the first plant portion, which is tolerant or resistant to the inhibitor, but does inhibit the same enzymatic activity in the second plant portion that is catalyzed by an enzyme sensitive to the selective inhibitor.

The present invention is further directed to a kit useful in methods for detecting and/or separating a plant portion of a first plant from a plant portion of a second plant in a mixture thereof, wherein plant portions of the first plant comprise a distinguishable marker, which marker is an enzyme. The kit comprises a detection agent and at least one compound selected from the group consisting of a surfactant, a selective inhibitor of an enzymatic activity present in plant portions of the second plant, and combinations thereof. Again, the selective inhibitor does not substantially inhibit the marker enzyme present in the first plant portion, which is tolerant or resistant to the inhibitor, but does inhibit the same enzymatic activity in the second plant portion that is catalyzed by an enzyme sensitive to the selective inhibitor.

### 4. DETAILED DESCRIPITION OF THE INVENTION

As used herein, the phrase “genetically modified” plant encompasses, but is not limited to, a plant that has been genetically altered using recombinant methodology. That is, the phrase “genetically modified plant” also refers to a plant that has been genetically altered using methodology not involving recombinant DNA technology including, but not limited to, crosses between plants to provide progeny carrying a genetic modification of a parent strain, where that genetic modification occurred spontaneously or was introduced by exposure to a mutagen.

The invention is directed toward compositions and methods for detecting plants, or portions thereof, that comprise a distinguishable marker. In certain methods of the present invention, the plant or plant portion is contacted with a detection agent that interacts with the distinguishable marker to provide a detectable signal.

In certain embodiments of the present invention, the distinguishable marker is an enzyme, and the detection agent or product derived therefrom comprises a chromogen, fluorophore, or luminescent moiety. In certain aspects of the present invention, the product derived from the exogenous detection agent is visible light.

In other embodiments of the present invention, the distinguishable marker is detectable marker and is identified in the absence of an exogenously-provided agent, such as a detection agent. In one aspects of this embodiment, the product, which is derived from at least one endogenous substrate, is visible light.

The plant portion corresponds to any of the parts of the plant, including an intact plant, such as but not limited to roots, tubers, berries, rhizomes, stems, leaves, flowers, shoots, seeds, fruits, grains, and seeds. In certain embodiments, the plant portion comprises a seed. In preferred embodiments, the portion is the seed portion of the plant.

In certain embodiments of the present invention, the plant or plant portion is contacted with a detection agent without substantially altering the viability or toxicity of the plant or portion thereof.

In certain embodiments, the present invention is also directed toward methods for identifying, monitoring, and/or separating specific non-genetically modified seeds from a mixture of seeds comprising either or both non-genetically modified seeds and genetically modified seeds. The methods disclosed herein comprise labeling specific genetically-modified seeds present in such mixtures, identifying those labeled, genetically-modified seeds using manual or automated detection means, and separating those labeled, genetically-modified seeds from the mixture using manual or automated separation means.

In certain embodiments of the present invention, the distinguishable marker of the genetically-modified seed is a protein, which can be an enzyme, that is expressed in the seed, and more preferably, expressed on the outer surface of the seed coat. Methods for directing a protein, generally expressed from a transgene, to the seed, and more particularly, the surface of the seed coat are known to those of ordinary skill in the art of plant genetic engineering. For example, oil-body proteins (“oleosins”) have been shown to function as carrier proteins in the construction of fusion proteins expressed in seeds from a recombinant gene (vanRooijen et al. 1995 BIO/TECHNOLOGY 13: 72-77). A fused protein, β-glucosidase, was shown to be expressed as part of the fusion protein, at high levels in seeds of Brassica napus. Moreover, there was no appreciable change in the level of β-glucosidase activity that could be extracted from such seeds expressing this fusion protein after storage at 4° C., under dry conditions, for more than one year.

In addition, proteins have been identified that adhere to the seed surface including, but not limited to the hydrophobic protein (HPS) of soybean (Glycine max [L.] Merr.). The HPS protein is highly expressed in the endocarp and adheres to the seed surface during development. The HPS gene is not expressed in the flower, leaf, embryo, stem, or root (Gizen et al. 1999 Plant Physiology 120: 951-50).

Therefore, in certain embodiments of the present invention, the distinguishable marker is a protein expressed as a fusion protein using oleosin or HPS as a carrier protein. In other embodiments, the distinguishable marker is a protein and is expressed from a recombinant gene comprising the structural gene for the marker, and the promoter, leader and termination signals of the gene encoding HPS. Similarly, other proteins of the outer surface of the seed coat are readily isolated and the genes encoding such proteins are readily identified, isolated, characterized, and engineered for the purposes of the present invention, using the methods disclosed in Gizen et al. Id., and the references cited therein.

The plant or plant portion to be analyzed is contacted, in certain embodiments, with a composition comprising the detection agent and a molecule, such but not limited to a surfactant, that can facilitate the interaction between the marker enzyme and the detection agent, which is a substrate of the marker enzyme.

In certain embodiments, the distinguishable marker is an enzyme, which can be selected from, but not limited to, the group consisting of β-D-glucuronidase, acetolactate synthase, dihydroflavonol reductase, flavonoid 3p 5p hydroxylase, neomycin phosphotransferase II, nopaline synthase, β-lactamase, phosphonothricin N-acetyltransferase, 5-enolpyruvylshikimate-3-phosphate synthase, glyphosate-resistant 5-enolpyruvylshikimate-3-phosphate synthase, glyphosate oxidoreductase, barnase ribonuclease, acetyl CoA carboxylase, DNA adenine methyl transferase, S-adenosylmethionine hydrolase, aminocyclopropane cyclase synthase, thioesterase, helicase, bromoxynil nitrilase, replicase (RNA-dependent RNA polymerase), and Δ-1, 2 desaturase.

Where the marker is an enzyme, the detection agent can be a substrate comprising a moiety such that cleavage or other modification of the substrate by the enzyme provides a product that is fluorescent, chemiluminescent, or chromogenic. Examples include, but are not limited to, 5-bromo-4-chloro-3-indolyl-phenylphosphonate (Dotson et al. Plant J. 1996 10(2): 383-92) and 5-bromo-4-chloro-3-indolyl-β-D-glucuronide (X-GUS) (Molecular Probes, Eugene, Oreg.). An example of a sensitive substrate for ribonuclease has been described (Kelemen et al. 1999 Nucleic Acids Research 27(18): 3696-3701). This ribonuclease substrate is a tetranucleotide, 5′-dArUdAdA-3′, comprising a 6-carboxyfluorescein moiety (6-FAM) attached to the 5′-terminus and a 6-carboxytetramethylrhodamine (6-TAMRA) moiety attached to the 3′-terminus of the tetranucleotide. Fluorescence of the 5′-(6-FAM) moiety is quenched by the proximal 3′-(6-TAMRA) moiety. Cleavage of this substrate by RNAase A, which physically separates the 5′ and 3′ moieties, resulted in a 180-fold increase in fluorescence.

The design and synthesis of specific enzyme substrates that comprise a chromogenic or fluorescent moiety and that yield a detectable reaction product are well known in the art or, where novel substrates/detection agents are identified, are readily adapted from the teaching of that art by one of ordinary skill. Such designs and syntheses include, but are not limited to: (a) fluorogenic and chromogenic β-lactamase substrates (U.S. Pat. No. 5,583,217); (b) chromogenic substrates of microbial enzymes (U.S. Pat. No. 6,051,391); (c) lipophilic fluorogenic glycoside substrates detectable at long wavelength (U.S. Pat. No. 5,242,805); (d) lipophilic fluorescent glycosidase substrates (U.S. Pat. No. 5,208,148); (e) chromogenic substrates for β-galactosidase and/or β-glucuronidase for identifying and differentiating bacterial species; (f) enzyme substrates that, upon cleavage, yield fluorescent precipitates (U.S. Pat. No. 5,316,906); (g) enzyme substrates or agents, for the detection of esterases and proteases (U.S. Pat. No. 4,758,508); (h) chromogenic dibenzoxasepinone and dibenzothiazepinone enzyme substrates (U.S. Pat. No. 5,3183,743); (i) chromogenic acridinone enzyme substrates (U.S. Pat. No. 4,810,636); and chromogenic merocyanine enzyme substrates (U.S. Pat. No. 5,191,073); (each of these patents is hereby incorporated by reference in its entirety).

In certain embodiments of the present invention, the chemically altered detection agent provides a colorimetric and/or fluorescent signal that is sufficiently different from that provided by the unaltered detection agent to enable the identification, monitoring, and separation of plant portions according the methods disclosed herein. Accordingly, in certain aspects of this embodiment, the detection agent is substantially colorless and/or is substantially non-fluorescent.

In other embodiments of the methods of the present invention, the marker is a distinguishable moiety, e.g., a protein that is, per se, readily detected and used to identify, for example, genetically modified seeds in a mixture. Such proteins or protein sets include but are not limited to green fluorescent protein, firefly luciferase, and a fusion protein comprising the luxA and luxB gene products of the Vibrio harveyi bacterial luciferase (see for example Kirchner et al. 1989 Gene 81(2): 349-54; Olsson et al. 1990 J. Biolumin. Chemilumin. 5(2): 79-87; Langridge et al. 1998 Methods Mol. Biol. 82: 385-96; Hanson et al. 2001 J. Exp. Bot. 53(356): 529-39; Zhang et al. 2001 Mol. Biotechnol. 17(2): 109-17).

In one aspect of this embodiment, the marker is a fluorescent protein, such as, but not limited to the green fluorescent protein of Aequorea victoria, or a derivative thereof with enhanced fluorescence in plant tissue, e.g., the engineered protein disclosed by Chiu et al. (Chiu et al. 1996, Current Biology 6 (3): 325-30, which is hereby incorporated by reference in its entirety). In another aspect of this embodiment, the marker is firefly luciferase or bacterial luciferase.

In another embodiment, the marker is at least a portion of a biosynthetic pathway that provides a detectable signal either in the absence or, in certain aspects of this embodiment, in the presence of an exogenous detection agent. In one, non-limiting example, the biosynthetic pathway is that of bacterial luciferase that is encoded by the lux genes of Vibrio fischeri or Vibrio harveyi. All or a portion of a biosynthetic pathway for bacterial luciferase can be expressed using e.g. lux genes of Vibrio fischeri or Vibrio harveyi transcribed from a heterologous promoter, thereby providing detectable signal, i.e., light (see, for example Engebrecht et al. 1985, Science 227 (4692): 1345-47, and Langridge et al. 1994, J. Biolumin. Chemilumin 9: 185-200, both of which are hereby incorporated by reference in their entirety). With respect to the lux genes of Vibrio fischeri, regulated expression of the lux operon can be achieved in the genetically-modified plant at a desired level, developmental stage, and in a particular tissue using methods, vectors, and reagents well known to those of ordinary skill in the art. In this aspect, the genetically engineered plant would produce n-decanal, the substrate for the luxA and luxB gene products. Alternatively, the genetically-modified plant or plant portion expresses only the luxA and luxB gene products. In this instance, a detection agent, n-decanal, can be applied to provide identification of genetically-modified plants or portions thereof expressing luxA and luxB gene products. In this aspect of the present invention, application of the detection agent and detection of plant portions elaborating light are sufficiently closely linked, temporally, to permit efficient and accurate identification, monitoring, and separation of plant portions providing this signal.

In certain embodiments, the distinguishable marker is expressed from a transgene, which is genetically engineered to determine the level, the timing, and the tissue specificity of expression of the transgene. In certain aspects of the methods of the present invention, the transgene is expressed under the control of one or more constitutive promoters (see, e.g. Li et al. 2001 Plant Sci. 160(5): 877-87), or from one or more promoters regulated by small-molecule effectors such as, but not limited to, galactose or galactosides (Bringhurst et al. 2001, 98(8): 4540-45), and tetracycline or tetracycline derivatives such as anhydrotetracycline (Weinmann et al. 1994 Plant J. 5(4): 559-69; Gossen et al. 1994 Curr. Opin. Biotechnol. 5(5): 516-20; and David et al. 2001 Plant Physiol. 125(4): 1548-53), as well as, e.g., registered agrochemicals such as RH5992 (Zuo et al. 2000, 11(2): 146-51). In other aspects of this embodiment, tissue-specific expression is achieved through the construction of chimeric genes comprising a tissue-specific expression system and a structural gene coding sequence comprising the coding sequence of a marker protein or a fusion protein comprising all or a portion of a marker protein (see, e.g., Gizen et al. 1999 Plant Physiology 120: 951-59; vanRooijen et al. 1995 Bio/Technology 13: 72-77; Treacy et al. 1997 Plant Mol. Biol. 34 (4): 603-11; Truernit et al. 1995 Planta 196 (3): 564-70; Bevan et al. 1993 Philos Trans R Soc Lond B Biol Sci 342: 209-15; and Capone et al. 1991 Plant Mol Biol 16 (3): 427-36). As noted above, in preferred embodiments, the marker protein is expressed as part of the seed coat, and in more preferred embodiments, the marker protein is expressed on the surface of the seed coat.

In certain embodiments, the marker is present only in one component of a mixture of plant portions, such as but not limited to a seed mixture to be monitored and/or sorted. However, in other embodiments, the marker gene can be present in more than one component of the seed mixture, but is nevertheless a distinguishing marker if its level of expression and/or the tissue-specificity of its expression in a first strain of seeds is sufficiently different from that of other seeds in the mixture, including, for example, non-genetically-modified seeds. In another aspect of this embodiment, the seed mixture is contacted with a composition comprising a detection agent and an agent that selectively inhibits the activity of the marker enzyme in one component of the mixture but not another. In still another aspect of this embodiment, the marker provides a detectable signal in the absence of an exogenously-added detection agent.

In another embodiment of the invention, a genetically-modified seed can comprise a transgene expressing anti-RNA that inhibits expression of one or more genes, thereby functionally removing a protein or carbohydrate, as non-limiting examples, from such genetically modified seed (see, e.g., Nakamura et al. 1996, Biosci. Biotechnol. Biochem. 60 (8): 1215-21). In this embodiment, non-genetically modified seed will comprise the distinguishing marker that is not present in the genetically-modified seed. In this embodiment, the marker can be an enzyme, and upon contacting a mixture of seeds comprising genetically-modified and non-genetically modified seeds with, e.g., a chromogenic substrate, non-genetically modified seeds will be labeled, identified and separated from the mixture.

In further embodiments of the present invention, a plant portion can comprise more than one marker allowing one stringent selection step or two separate signals for two, serial or concurrent separations.

The distinguishable marker can be responsible for a valuable trait conferred upon the transgenic plant. Such traits include, but are not limited to resistance to specific herbicides, resistance to undesirable insects, or acquisition of new chemical qualities such as production of new high-value oils. For example, the enzymes acetolactate synthase, dihydroflavonol reductase, flavonoid 3p 5p hydroxylase, nopaline synthase, phosphonothricin N-acetyl transferase, 5-enolpyruvylshikimate-3-phosphate synthase, glyphosate-resistant 5-enolpyruvylshikimate-3-phosphate synthase, glyphosate oxidoreductase, barnase ribonuclease, acetyl CoA carboxylase, bromoxynil nitrilase and Δ-1, 2 desaturase, each confer a value-added trait to their respective host organism. These traits include a change in color, production of high-value oil, resistance to pests, and resistance to strategic herbicides.

The distinguishable marker can be responsible for a trait utilized in laboratory production of the genetically-modified plant portion. Such traits include resistance to specific herbicides, or acquisition of new enzymatic activities. One specific, non-limiting example is resistance to the herbicide glyphosate. Another specific example is acquisition of the enzymatic ability to modify (e.g. phosphorylate, acetylate, or adenylylate) and/or inactivate aminoglycoside antibiotics such as but not limited to neomycin.

The marker can be a null allele in the transgenic plant removing for example an enzyme of the seed, grain, fruit etc. that is normally found in non-genetically-modified-plant. In one, non-limiting aspect of this embodiment, the null allele results in a change in color of the genetically-modified plant and/or portions thereof. The distinguishable marker can also be a mutant allele in the transgenic plant altering an enzyme of the seed, grain, fruit etc. to provide a distinguishing trait that is detected by a detection agent that does not react appreciably in non-genetically-modified seeds of the parent strain or cultivar.

In sorting mixtures, for example the genetically-modified-material is “rejected” based upon a first signal generated while the non-genetically-modified-material is carried along the conveyer. At another point along the conveyer, a second signal, unique to the non-genetically-modified-material is generated allowing the non-genetically-modified-material to be positively identified and separated from the remaining material and collected separately. Repeated cycles allow isolation of e.g. pure stocks of both genetically-modified-material as well as non-genetically-modified material. Variations include mixtures of detection agents specific to each of the plant components to be isolated, where each detection agent for example comprises a different detection signal.

In certain embodiments of the present invention, plants or plant portions, which can be, but are not limited to, labeled seeds are identified using automated detection means, and separated using automated separation means in commercially available processing equipment employing automated inspection technology. Generally, such processing equipment comprises a conveyer system for transporting material to be sorted into a detection area that includes a color imaging system for signal detection. The imaging system is coupled to an ejection system through a computer-controlled linkage, whereby individual particles that have been identified as meeting pre-determined criteria are removed from the conveyer system, generally with a milliseconds-long burst of compressed air.

The color imaging system can include, without limitation, a plurality of charge-coupled-device (CCD) cameras capable of detecting colored or fluorescent areas that are less than or equal to about 0.3 mm in diameter, and that are capable of 24 bit RGB color image processing (theoretically capable of recognizing 2²⁴, i.e. 16,777,216 colors). The colored or fluorescent area may include the entire plant or plant portion or may include only a small region of the plant or plant portion. In certain embodiments, the colored or fluorescent region of the plant or plant portion is less than or equal to about 0.3 mm in diameter. Where the label is fluorescent, suitable detection systems also include a light source emitting light of the appropriate wavelength for absorption by the fluorescent molecule or moiety, as well as a detector capable of detecting the light emitted by the fluorescent label.

The ejector system generally comprises a plurality of ejector modules that are attached to a high pressure air source. Each ejector module comprises a computer-controlled valve or gate positioned in close proximity to the position on the conveyer system where samples are identified by the detection system. The image of the plant or plant portion, such as but not limited to a seed is recorded by the color imaging system and compared to user-defined criteria incorporated into the computer software. Where the tested sample meets the software-specified criteria, a signal is sent from the computer to the ejector means to open the valve or gate for a short period of time, usually less than 10 milliseconds, releasing a burst of pressurized air sufficient to remove the particle from the conveyer. In other embodiments, the ejected seed is collected separately from the rest of the mixture which continues to be transported by the conveyer system and, ultimately, collected.

In certain embodiments of the present invention, the mixture of plant portions to be sorted includes genetically modified plant portions that comprises the distinguishing marker. In this embodiment, using automated equipment as described above, the genetically modified plant portion is labeled, identified, detected, and sorted (i.e. “ejected”) from the automatically conveyed mixture. In preferred embodiments, the sorted, non-genetically modified plant portion contains less than about 10% genetically-modified plant portions, in more preferred embodiments, less than about 5% genetically-modified plant portions, more preferably, less than about 2% genetically-modified plant portions, and most preferably, less than 1% genetically-modified plant portions. In other embodiments, purified, non-genetically modified plant portions are subjected to further cycles of purification according to the present invention to provide non-genetically-modified plant portions comprising 0.1% or less genetically-modified plant portions. In a specific aspect of this embodiment, the plant portion is a seed.

Non-limiting examples of commercially available sorting equipment suitable for use in the present invention, provided appropriate colorimetric and/or fluorescence-detection modules are installed, include but are not to be limited to: SCAN MASTER (Satake Corporation, Houston, Tex.), IGUAZU-PENTA and IGUAZU-WORLDSORTER (Delta Technology Corporation, Houston, Tex.), NIAGARA (Sortex, Stockton, Calif.), and TEGRA (Key Technology, Inc., Walla Walla, Wash.). Representative examples of suitable colorimetric and/or fluorescence-detection modules include, but are not limited to the Tegra Vis/IR, trichromatic, monochromatic, and ultraviolet lamp options (Key Technology, Inc., Walla Walla Wash.).

In one aspect of the present invention, the mixture of plants and/or plant portions to be identified, monitored, and/or sorted comprises genetically-modified plant portions carrying the genetic marker for tolerance to the herbicide glyphosate (N-phosphonomethyl glycine). Commercially valuable crop plants comprising this marker include, but are not limited to, soybeans, corn, tobacco, and sugar beets.

Glyphosate inhibits the enzyme 5-enolpyruvylshikimate 3-phosphate synthase (“EPSP synthase”), which is involved in aromatic amino acid biosynthesis and catalyzes the following reaction (See, e.g., Alibhai et al. 2001 Proc. Natl. Acad. Sci. USA 98 (6): 2944-46 and Schonbrunn et al. 2001 Proc. Natl. Acad. Sci. USA 98 (4): 1376-80, each of which is hereby incorporated by reference in its entirety):

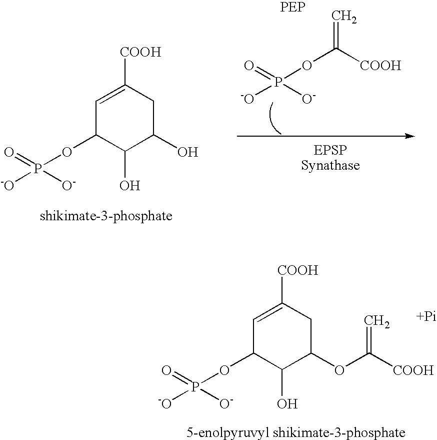

Based upon X-ray crystallographic analysis it has been reported that glyphosate appears to occupy the phosphoenolpyruvate (“PEP”) binding site of EPSP synthase. Consistent with this assertion is the observation that a glyphosate tolerant EPSP synthase was identified as having an amino acid substitution, (in which the glycine at position 96 is replaced by an alanine, “G96A,” in the glyphosate-tolerant mutant), that provides a methyl group that, based upon the structural analysis reported, would interfere more strongly with glyphosate binding than with phosphoenolpyruvate binding (Schonbrunn et al. 2001, Proc. Natl. Acad. Sci. USA. 98(4): 1376-80, which is hereby incorporated by reference in its entirety).

Plant genes encoding glyphosate-tolerant EPSP synthase have been identified, isolated, and introduced into different, agronomically-important plants to provide genetically-modified glyphosate-tolerant plants. In another approach, bacterial genes encoding kinetically-efficient, glyphosate-tolerant EPSP synthases have been identified, isolated, and expressed in agronomically-important plants to provide genetically-modified glyphosate-tolerant strains. (See for example, U.S. Pat. Nos. 5,663,435, 6,225,114 B1, and 6,248,867 B1, each of which is incorporated herein by reference in its entirety).

Accordingly, such genetically-modified glyphosate tolerant plants or plant parts can be detected, selectively, within a mixture of EPSP synthase-expressing plants or plant parts by assaying for EPSP synthase activity in the presence of glyphosate. In one embodiment of the present invention, this assay is carried out by contacting the mixture of EPSP synthase-expressing plants or plant parts with a composition comprising a detection reagent of the present invention, such that the following enzymatic reaction occurs, releasing R, which is a chromogenic or fluorescent molecule.

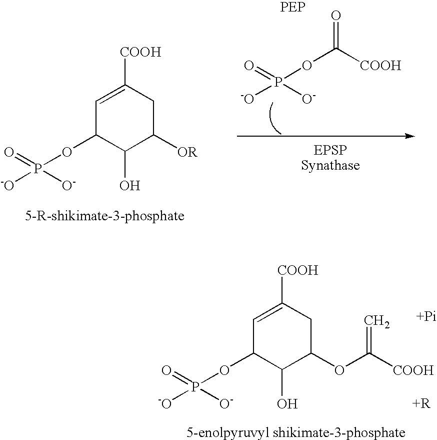

In certain embodiments, the fluorogenic or chromogenic moiety, R, is selected from the following group of molecules and attached, according to methods well known in or readily adapted from the art, to the enzyme substrate, shikimate-3-phosphate, to provide a detection agent useful in the present invention.

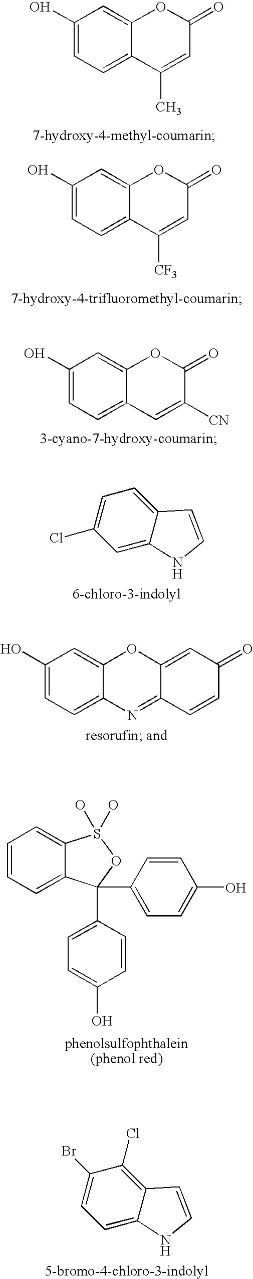

Accordingly, representative, but non-limiting examples of detection agents useful in this embodiment of the present invention include the following:

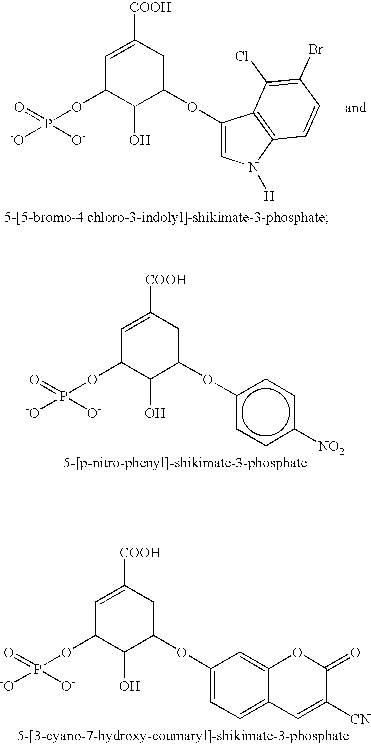

Suitable methods that can be used for the synthesis of detection agents useful in this embodiment of the present invention, as well as alternative fluorogenic or chromogenic moieties that may be attached to shikimate-3-phosphate to provide detection agents useful in this embodiment of the present invention, include but are not limited to those described in U.S. Pat. Nos. 5,583,217, 6,051,391, 5,242,805, 5,208,148, 5,316,906, 5,316,906, 4,758,508, 5,3183,743, 4,810,636, and 5,191,073, each of which is hereby incorporated by reference in its entirety.

In certain embodiments of the present invention, it is advantageous to include a molecule, such as but not limited to, a surfactant, in compositions comprising a detection agent of the present invention, to facilitate or enhance the interaction between the marker in the plant or plant portion contacted by the detection agent. In a specific embodiment, the composition also includes a selective inhibitor (such as but not limited to glyphosate) of the marker in the genetically-modified plant or plant portion or the non-genetically-modified plant or plant portion. Formulations suitable for the compositions of the present invention that can be used for application of a detection agent to plants, plant portions and mixtures of different plants or plant portions, are well-known to those in the art and include, but are not limited, to those described in U.S. Pat. Nos. 5,317,003, 5,703,016, 5,565,409, 5,693,593, 6,127,317, 6,228,807 B1, 6,277,788 B1, as well as those described in European Patent Application no. 220,902 A2, each of which is hereby incorporated herein by reference in its entirety.

In another embodiment of the present invention, the mixture of plants and/or plant portions to be identified, monitored, and/or sorted comprises genetically-modified plant portions carrying a highly-expressed, heterologous gene encoding a thioesterase involved in fatty acid biosynthesis (e.g. the 12:0 ACP thioesterase from the California Bay tree, Umbellularia califomica). Expression, and more particularly over-expression, of such a thioesterase in the agronomically-important canola plant, Brassica napus (Argentine canola) results in an improved, advantageous balance of esterified fatty acids in the triglycerides of that plant. That is, canola oil isolated from genetically-modified plants overexpressing such a heterologous thioesterase has an increased level of lauric and myristic acid, and a decreased level of oleic, linoleic, and palmitic acids. In preferred embodiments, the thioesterase gene is expressed from a seed-specific promoter, thereby affecting the fatty acid content and distribution of the oil present in canola seeds.

Accordingly, plants and/or plant portions, including seeds, of a plant genetically modified to overexpress such a heterologous thioesterase will have a detectably-higher level of that enzymatic activity than other plants or plant portions that have not been genetically-modified with respect to this trait. Therefore, assay of plants, plant portions, and mixtures of plants or plant portions that comprise genetically modified plants or plant portions comprising a heterologous, overexpressed thioesterase can be detected, monitored, and sorted using the compositions and methods of the present invention.

More specifically, a thioesterase (TE) catalyzes the following reaction:

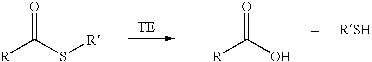

Therefore, where R'SH is a chromogenic or fluorescent molecule, the presence of a genetically-modified plant or plant portion over-expressing a heterologous thioesterase is readily detected according to the present invention. Two non-limiting examples of detection agents useful for the detection of thioesterase activity in plants or portions thereof are as follows:

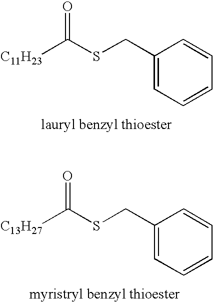

Hydrolysis of each of these substrates/detection agents with a thioesterase releases the corresponding fatty acid (lauric or myristic acid respectively) and a benzyl thiol, which can be detected, for example, by reaction with thiol reagent such as, but not limited to 5,5′-dithiobis(2-nitrobenzoic acid) (i.e. DTNB or Elman's reagent) yielding a product having a Molar extinction coefficient of 13,260 at 405 nm; or by reaction with 4,4′-dithiopyridine, yielding a 4-thiopyridone product having a Molar extinction coefficient of 19,800 at 324 nm.

Suitable detection agents for identifying, monitoring, and/or separating plants or plant portions comprising an overexpressed heterologous thioesterase in a genetically-modified plant or plant portion, are synthesized according to the following general scheme:

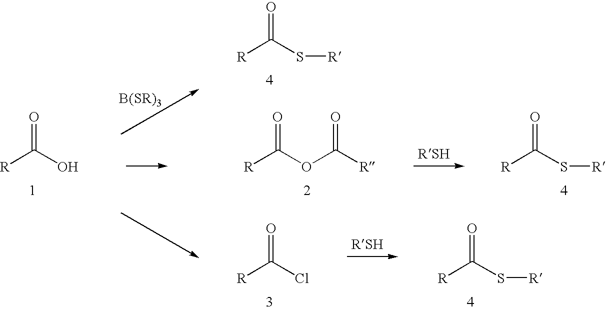

A carboxylic acid 1 may be elaborated to the thioester 4 directly via the agency of B(SR)₃ (1977 J. Chem. Soc., Perkin Trans. 1, 1672) or through the intermediacy of either the anhydride 2 (1986 Tetrahedron Lett. 27: 3791) or the acyl chloride 3 (1979 Top. Sulfur Chem. 4: 1-373). Alternately, an ester 5 may be directly converted to the thioester 4 by treatment with trimethylsilyl sulfides and AlCl₃ (J. Org. Chem. 1977, 42: 3960).

A further embodiment of the present invention is directed toward the detection, monitoring, and/or sorting of genetically-modified plants or plant portions comprising the enzyme β-glucuronidase. A gene encoding this enzyme can be incorporated into and expressed in a genetically-modified plant or plant portion thereby providing a marker useful in the present invention. Therefore a plant, plant portion, or a mixture comprising a plant or plant portion expressing β-glucuronidase can be detected using the methods of the present invention, allowing the detection and separation e.g. of genetically-modified plants or portions thereof from a mixture of plants or plant portions comprising plants or plant portions that have not been modified to express this marker enzyme. Numerous detection agents suitable for use in this embodiment of the present invention, as well as methods for their synthesis, have been disclosed in the art, including but not limited to U.S. Pat. Nos. 5,242,805, 5,316,906, 5,208,148, 5,358,854, 4,810,636, 5,191,073, 5,183,743, and 5,358,854, each of which is hereby incorporated herein by reference in its entirety.

In a still further embodiment of the present invention, genetically-modified plants or plant portions expressing the enzyme 1-amino-cyclopropane-1-carboxylic acid deaminase (ACCd) (e.g. from Pseudomonas chlororaphis) are detected, monitored, and/or separated according to the methods of the present invention. The enzyme ACCd deaminates 1-amino-cyclopropane-1-carboxylic acid, which is an essential precursor of ethylene that is required for ripening of fruit, including tomatoes, according to the following reaction:

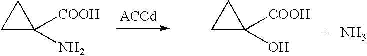

Expression of this heterologous enzyme, ACCd, in plants or portions thereof, e.g. fruit, delays the ripening process by decreasing the level of ethylene in the plant or plant portion, thereby providing an agronomic advantage to such genetically-modified plants or plant portions. Identification, monitoring, and separating plants or plant portions expressing ACCd therefore, can be carried out according the methods of the present invention by contacting the plant or portion thereof with a detection agent formed by joining a detectable moiety to a substrate of ACCd such that cleavage of the substantially colorless and/or non-fluorescent detection agent will provide a detectable reaction product. One non-limiting example of such a detection agent, and the reaction products generated by ACCd deamination, are shown below:

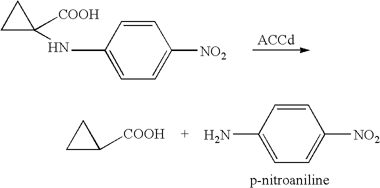

One, non-limiting, method for the synthesis of a suitable detection agent is as follows:

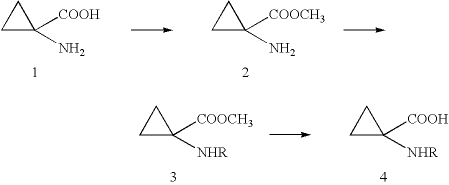

According to this method, commercially available 1-amino-cyclopropane-1-carboxylic acid 1 is protected as the methyl ester 2 via esterification (e.g. with diazomethane). The amino group is then alkylated to yield a secondary amino ester 3 which is then hydrolyzed to afford the desired ACC analog 4. N-alkylation procedures suitable for conversion of 2 to 3 are well known in the art and include, but are not limited to those disclosed in Larock, R. C., Comprehensive Organic Transformations—A Guide To Functional Group Preparations, 1989, pp. 401-402, which is hereby incorporated by reference in its entirety.

The present invention further encompasses compositions useful in methods for detecting and/or separating a plant portion of a first plant from a plant portion of a second plant in a mixture thereof, wherein plant portions of the first plant comprise a distinguishable marker, which marker is an enzyme. Such compositions include, but are not limited to those comprising a detection agent and at least one compound selected from the group consisting of a surfactant and a selective inhibitor of an enzymatic activity present in plant portions of said second plant, as well as combinations thereof.

In certain embodiments, such compositions are useful in separation of plant portions from mixtures of plant portions as disclosed herein, where the distinguishable marker is glyphosate-resistant 5-enolpyruvylshikimate-3-phosphate synthase. In one aspect of such embodiments, the selective inhibitor is glyphosate.

Such compositions may comprise, but are not limited to, those comprising a detection agent selected from the group consisting of:

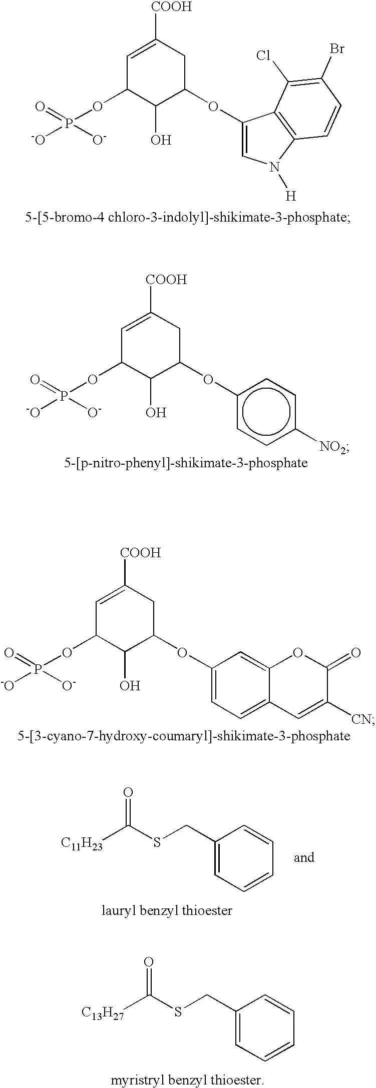

The present invention is also directed to kits useful in methods for detecting and/or separating a plant portion of a first plant from a plant portion of a second plant in a mixture thereof, where plant portions of the first plant comprise a distinguishable marker, which marker is an enzyme. Kits according to the present invention comprise a detection agent and at least one compound selected from the group consisting of a surfactant, a selective inhibitor, and combinations thereof. The selective inhibitor does not substantially inhibit the marker enzyme present in the first plant portion, which is tolerant or resistant to the inhibitor, but does inhibit the same enzymatic activity in the second plant portion that is catalyzed by an enzyme sensitive to the selective inhibitor. For example, in a specific embodiment, plant portion of the first plant comprise a glyphosate-tolerant or glyphosate-resistant 5-enolpyruvylshikimate-3-phosphate synthase while plant portions of the second plant comprise a glyphosate-sensitive 5-enolpyruvylshikimate-3-phosphate synthase, and the kit comprises a selective inhibitor, glyphosate. In a specific embodiment, kits of the present invention are useful for detecting, monitoring and separating seeds of a first plant from seeds of a second plant present in a mixture thereof.

Kits of the present invention include those comprising, but not limited to, a detection agent selected from the group consisting of

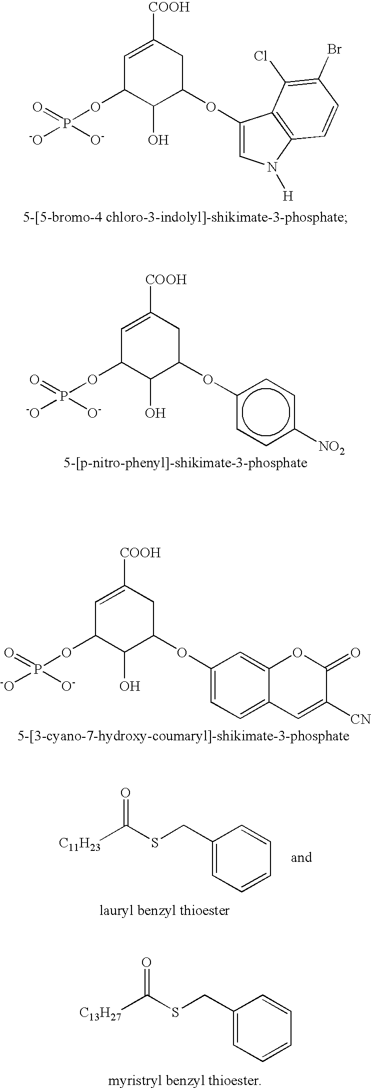

### 5. EXAMAPLE

### Labeling of Genetically-Modified Soybean Seeds Expressing Beta-Glucuronidase from a Transgene

A mixture of seeds comprising genetically-modified soybean seeds expressing transgenic β-glucuronidase that is associated with the seed coat is contacted with a detection reagent comprising a chromogenic substrate for β-glucuronidase, 5-bromo-4-chloro-3-indolyl-β-D-glucuronide (“X-GLUC”) (Molecular Probes, Eugene, Oreg.).

The detection reagent is formulated by dissolving 5 mg X-GLUC in 0.05 mL N,N,-dimethyl formamide and adding this solution to 10 mL of 0.05 M NaPO₄, pH 7, and is stored at 4° C. until used. A sufficient volume of detection reagent is added to the mixture to cover the seeds, which are then incubated overnight at 37° C.

The detection reagent is removed by aspiration, and the seeds are covered with a solution, designated FAA. FAA is formulated by adding 10 mL formaldehyde, 10 mL of acetic acid, and 75 mL of ethanol to 105 mL of water, and is also stored at 4° C. until use. After 10 minutes of incubation at room temperature, FAA is removed, and the seed mixture is incubated for two minutes in 50% ethanol, two minutes in 100% ethanol, and for minute in water. The seed mixture is observed visually, and those seeds exhibiting a blue color are separated from the mixture by hand.

### 6. EXAMPLE Labeling of Genetically-Modified Mustard Seeds Expressing Beta-Glucuronidase from a Transgene

A mixture of seeds comprising genetically-modified Arabidopsis seeds expressing transgenic β-glucuronidase, (Arabidopsis Biological Resource Center, Ohio State University, Columbus, Ohio), is contacted with a detection reagent comprising a chromogenic substrate for β-glucuronidase, 5-bromo-4-chloro-3-indolyl-β-D-glucuronide (“X-GLUC”) (Molecular Probes, Eugene, Oreg.).

The detection reagent is formulated by dissolving 5 mg X-GLUC in 0.05 mL N,N,-dimethyl formamide and adding this solution to 10 mL of 0.05 M NaPO₄, pH 7, and is stored at 4° C. until used. A sufficient volume of detection reagent is added to the mixture to cover the seeds, which are then incubated overnight at 37° C.

The detection reagent is removed by aspiration, and the seeds are covered with a solution, designated FAA. FAA is formulating by adding 10 mL formaldehyde, 10 mL of acetic acid, and 75 mL of ethanol to 105 mL of water, and is also stored at 4° C. until use. After 10 minutes of incubation at room temperature, FAA is removed, and the seed mixture is incubated for two minutes in 50% ethanol, two minutes in 100% ethanol, and for minute in water. The seed mixture is observed visually, and those seeds exhibiting a blue color are separated from the mixture by hand.

Each reference cited herein is hereby incorporated by reference in its entirety for all purposes. The present invention is not to be limited by the scope of the specific embodiments described herein. Indeed, various modifications of the invention in addition to those described herein will become apparent to those of skill in the art from the foregoing description and accompanying figures. Such modifications are intended to fall within the scope of the appended claims.

1. A method for distinguishing a genetically engineered plant portion from a non-genetically engineered plant portion, the method comprising:
(a) providing a mixture of plant portions, wherein the mixture comprises a genetically-engineered plant portion and a non-genetically engineered plant portion, and wherein the genetically-modified plant portion comprises a distinguishable marker which marker is an enzyme;
(b) contacting said mixture with a composition comprising a detection agent under conditions and for a time sufficient for the enzyme to alter the detection agent chemically to provide a detectable product, which detectable product is associated with the genetically engineered plant portion, thereby generating a labeled plant portion; and
(c) identifying the labeled plant portion using automated detection means.

2. A method for separating a genetically engineered plant portion from a non-genetically engineered plant portion, the method comprising:
(a) providing a mixture of plant portions, wherein the mixture comprises a genetically engineered plant portion and a non-genetically engineered plant portion, and wherein the genetically engineered plant portion comprises a distinguishable marker which marker is an enzyme;
(b) contacting said mixture with a composition comprising a detection agent under conditions and for a time sufficient for the enzyme to alter the detection agent chemically to provide a detectable product, which detectable product is associated with the genetically engineered plant portion, thereby generating a labeled plant portion;
(c) identifying the labeled plant portion using automated detection means; and
(d) separating the labeled plant portion from the mixture using automated separation means.

3. A method for distinguishing a plant portion of a first plant from a plant portion of a second plant, the method comprising:
(a) providing a mixture of plant portions, wherein the mixture comprises a plant portion of a first plant and a plant portion of a second plant, and wherein the plant portion of the first plant comprises a distinguishable marker which marker is an enzyme;
(b) contacting said mixture with a composition comprising a detection agent under conditions and for a time sufficient for the enzyme to alter the detection agent chemically to provide a detectable product, which detectable product is associated with the plant portion of the first plant, thereby generating a labeled plant portion; and
(c) identifying the labeled plant portion using automated detection means.

4. A method for separating a plant portion of a first plant from a plant portion of a second plant, the method comprising:
(a) providing a mixture of plant portions, wherein the mixture comprises a plant portion of a first plant and a plant portion of a second plant, and wherein the plant portion of the first plant comprises a distinguishable marker which marker is an enzyme;
(b) contacting said mixture with a composition comprising a detection agent under conditions and for a time sufficient for the enzyme to alter the detection agent chemically to provide a detectable product, which detectable product is associated with the plant portion of the first plant, thereby generating a labeled plant portion;
(c) identifying the labeled plant portion using automated detection means; and
(d) separating the labeled plant portion from the mixture using automated separation means.

5. The method of claim 1, 2, 3, or 4, wherein the detection agent is substantially colorless.

6. The method of claim 1, 2, 3, or 4, wherein the detection agent is substantially non-fluorescent.

7. The method of claim 1, 2, 3, or 4, wherein said contacting is automated.

8. The method of claim 1, 2, 3, or 4, wherein the mixture of plant portions comprises metabolizing plant portions.

9. The method of claim 1, 2, 3, or 4, wherein the mixture of plant portions comprises harvested plant portions.

10. The method of claim 1, 2, 3, or 4, wherein the chemical alteration comprises cleavage of the detection agent.

11. The method of claim 10, wherein a cleavage product is a chromophoric, fluorescent, or chemiluminescent cleavage product.

12. The method of claim 1, 2, 3, or 4, wherein the chemical alteration comprises hydrolysis of the detection agent.

13. The method of claim 1, 2, 3, or 4, wherein the contacted mixture is not toxic to a mammal.

14. The method of claim 13, wherein the mammal is a human.

15. The method of claim 1, 2, 3, or 4, wherein food prepared from a plant portion of the contacted mixture is not toxic to humans.

16. The method of claim 1 or 2, wherein the genetically engineered plant portion and the non-genetically-modified plant portion are seeds.

17. The method of claim 16, wherein the viability of the genetically engineered seed and the viability of the non-genetically engineered seed are not substantially reduced.

18. The method of claim 3 or 4, wherein at least one of the plant portion of a first plant and the plant portion of the second plant is a plant portion of a genetically engineered plant.

19. The method of claim 3 or 4, wherein the plant portion of the first plant and the plant portion of the second plant are seeds.

20. The method of claim 19, wherein the viability of the seeds of the plant portions of the first plant and of the second plant are not substantially reduced.

21. The method of claim 1 or 2, wherein the genetically engineered plant portion comprising a marker is selected from the group consisting of corn, soybean, oat, rye, sunflower, wheat, rice, barley, beet, canola, cotton, potato, chicory, tomato, carnation, melon, tobacco, pea, mustard plant portions, and mixtures thereof.

22. The method of claim 3 or 4, wherein the plant portion comprising a marker is selected from the group consisting of corn, soybean, oat, rye, sunflower, wheat, rice, barley, beet, canola, cotton, potato, chicory, tomato, carnation, melon, tobacco, pea, mustard plant portions, and mixtures thereof.

23. The method of claim 1 or 2, wherein the genetically-modified plant portion is derived from a transgenic plant.

24. The method of claim 1 or 2, wherein the enzyme is selected from the group consisting of β-D-glucuronidase, acetolactate synthase, dihydroflavonol reductase, flavonoid 3p 5p hydroxylase, neomycin phosphotransferase II, nopaline synthase, β-D-glucuronidase, acetolactate synthase, dihydroflavonol reductase, flavonoid 3p 5p hydroxylase, neomycin phosphotransferase, neomycin phosphotransferase II, acetolactate synthase, nopaline synthase, β-lactamase, phosphonothricin N-acetyltransferase, 5-enolpyruvylshikimate-3-phosphate synthase, glyphosate-resistant 5-enolpyruvylshikimate-3-phosphate synthase, glyphosate oxidoreductase, barnase ribonuclease, acetyl CoA carboxylase, DNA adenine methyl transferase, S-adenosylmethionine hydrolase, aminocyclopropane cyclase synthase, thioesterase, helicase, bromoxynil nitrilase, replicase (RNA-dependent RNA polymerase), and Δ-12 desaturase.

25. The method of claim 1 or 2, wherein the enzyme is expressed from a transgene.

26. The method of claim 1, 2, 3, or 4, wherein the enzyme is glyphosate-resistant 5-enolpyruvylshikimate-3-phosphate synthase, and wherein the composition comprises glyphosate.

27. The method of claim 26, wherein the detection agent is selected from the group consisting of:

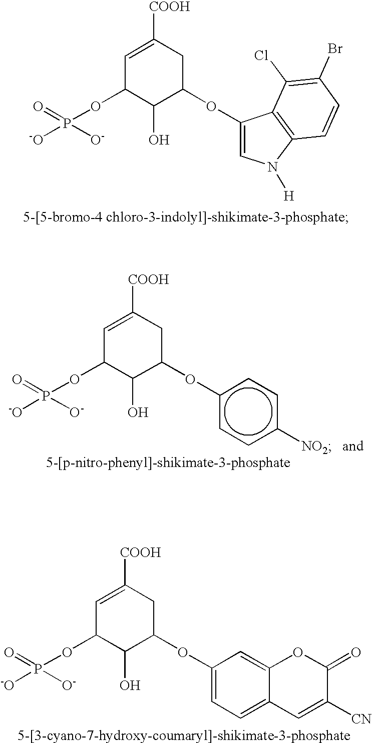

28. The method of claim 1 or 2, wherein the enzyme is β-glucuronidase.

29. The method of claim 1 or 2, wherein the enzyme is 12:0 ACP thioesterase.

30. The method of claim 29, wherein the detection agent is selected from the group consisting of:

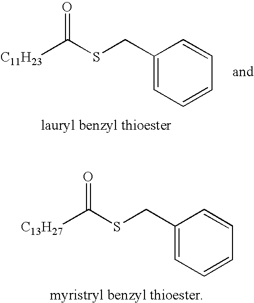

31. The method of claim 1 or 2, wherein the enzyme is 1-amino-cyclopropane-1-carboxylic acid deaminase.

32. A method for distinguishing a plant portion of a first plant from a plant portion of a second plant, the method comprising:
(a) providing a mixture of plant portions, wherein the mixture comprises a plant portion of a first plant and a plant portion of a second plant, wherein the plant portion of the first plant comprises a distinguishable marker which marker is a protein, wherein said protein provides a detectable signal in the absence of exogenous substrate, and wherein the detectable signal is associated with the plant portion of the first plant, thereby generating a labeled plant portion; and
(b) identifying the labeled plant portion using automated detection means.

33. A method for separating a plant portion of a first plant from a plant portion of a second plant, the method comprising:
(a) providing a mixture of plant portions, wherein the mixture comprises a plant portion of a first plant and a plant portion of a second plant, wherein the plant portion of the first plant comprises a distinguishable marker which marker is a protein, wherein said protein provides a detectable signal in the absence of exogenous substrate, and wherein the detectable signal is associated with the plant portion of the first plant, thereby generating a labeled plant portion;
(b) identifying the labeled plant portion using automated detection means; and
(c) separating the labeled plant portion from the mixture using automated separation means.

34. The method of claim 32 or 33, wherein at least one of the first plant and the second plant is a genetically engineered plant.

35. The method of claim 34, wherein the protein comprises at least a portion of a green fluorescent protein of Aequorea victoria.

36. The method of claim 35, wherein the green fluorescent protein of Aequorea victoria is optimized for expression in plants.

37. A method for distinguishing a plant portion of a first plant from a plant portion of a second plant, the method comprising:
(a) providing a mixture of plant portions, wherein the mixture comprises a plant portion of a first plant and a plant portion of a second plant, wherein the plant portion of the first plant comprises a distinguishable marker which marker comprises at least a portion of a biosynthetic pathway which provides a detectable signal in the absence of exogenous substrate, and wherein the detectable signal is associated with the plant portion of the first plant, thereby generating a labeled plant portion; and
(b) identifying the labeled plant portion using automated detection means.

38. A method for separating a plant portion of a first plant from a plant portion of a second plant, the method comprising:
(a) providing a mixture of plant portions, wherein the mixture comprises a plant portion of a first plant and a plant portion of a second plant, wherein the plant portion of the first plant comprises a distinguishable marker which marker comprises at least a portion of a biosynthetic pathway that provides a detectable signal in the absence of exogenous substrate, and wherein the detectable signal is associated with the plant portion of the first plant, thereby generating a labeled plant portion;
(b) identifying the labeled plant portion using automated detection means; and
(c) separating the labeled plant portion from the mixture using automated separation means.

39. The method of claim 37 or 38, wherein at least one of the first plant and the second plant is a genetically engineered plant.

40. The method of claim 39, wherein the biosynthetic pathway is a bacterial luciferase biosynthetic pathway selected from the group consisting of the lux biosynthetic pathway encoded by the lux operon of Vibrio fischeri and the lux biosynthetic pathway encoded by the lux operon of Vibrio harveyi.

41. The method of claim 40, wherein said pathway is optimized for expression in plants.

42. A method for distinguishing a plant portion of a first plant from a plant portion of a second plant, the method comprising:
(a) providing a mixture of plant portions, wherein the mixture comprises a plant portion of a first plant and a plant portion of a second plant, wherein the plant portion of the first plant comprises a distinguishable marker which marker comprises at least a portion of a biosynthetic pathway;
(b) contacting said mixture with a composition comprising a detection agent, wherein said detection agent is altered chemically by said pathway or portion thereof to provide a detectable signal, and wherein the detectable signal is associated with the plant portion of the first plant, thereby generating a labeled plant portion; and
(c) identifying the labeled plant portion using automated detection means.

43. A method for separating a plant portion of a first plant from a plant portion of a second plant, the method comprising:
(a) providing a mixture of plant portions, wherein the mixture comprises a plant portion of a first plant and a plant portion of a second plant, wherein the plant portion of the first plant comprises a distinguishable marker which marker comprises at least a portion of a biosynthetic pathway;
(b) contacting said mixture with a composition comprising a detection agent, wherein said detection agent is altered chemically by said pathway or portion thereof to provide a detectable signal, and wherein the detectable signal is associated with the plant portion of the first plant, thereby generating a labeled plant portion;
(c) identifying the labeled plant portion using automated detection means; and
(d) separating the labeled plant portion from the mixture using automated separation means.

44. The method of claim 42 or 43, wherein said contacting is automated.

45. The method of claim 42 or 43, wherein the detection agent is substantially colorless.

46. The method of claim 42 or 43, wherein the detection agent is substantially non-fluorescent.

47. The method of claim 42 or 43, wherein said biosynthetic pathway or portion thereof comprises a bacterial luciferase activity encoded by the luxA and luxB of Vibrio fischeri, said detection agent is decanal, and said detectable signal is visible light.

48. A composition useful in a method for detecting and/or separating a plant portion of a first plant from a plant portion of a second plant in a mixture thereof, wherein the plant portion of the first plant comprises a distinguishable marker, which marker is an enzyme, the composition comprising a detection agent and at least one compound selected from the group consisting of a surfactant and a selective inhibitor the enzyme present in plant portions of said second plant, and combinations thereof.

49. The composition of claim 48, wherein the distinguishable marker is glyphosate-resistant 5-enolpyruvylshikimate-3-phosphate synthase and the composition comprises a selective inhibitor, wherein the selective inhibitor is glyphosate.

50. The composition of claim 48, wherein the detection agent is selected from the group consisting of

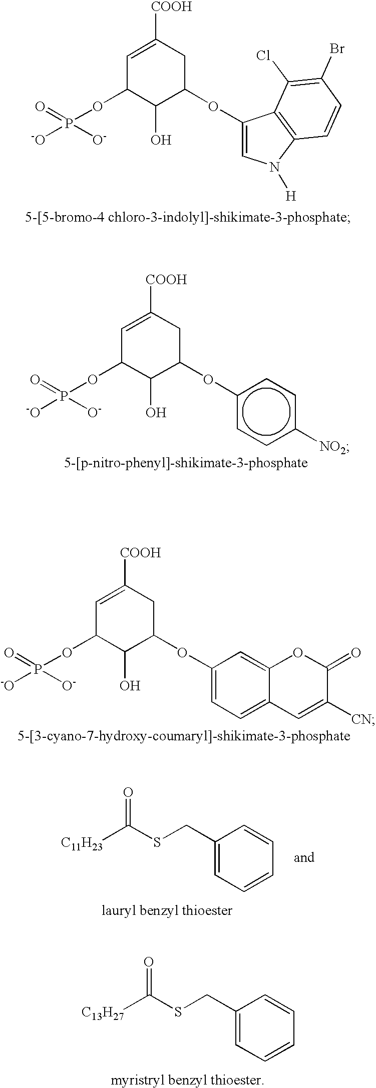

51. A kit useful in a method for detecting and/or separating a plant portion of a first plant from a plant portion of a second plant in a mixture thereof, wherein the plant portion of the first plant comprises a distinguishable marker, which marker is an enzyme, the kit comprising a detection agent and at least one compound selected from the group consisting of a surfactant, a selective inhibitor of the enzyme present in the plant portion of said second plant, and combinations thereof.

52. The kit of claim 51, comprising a selective inhibitor, wherein the selective inhibitor is glyphosate.

53. The kit of claim 51, wherein the detection agent is selected from the group consisting of

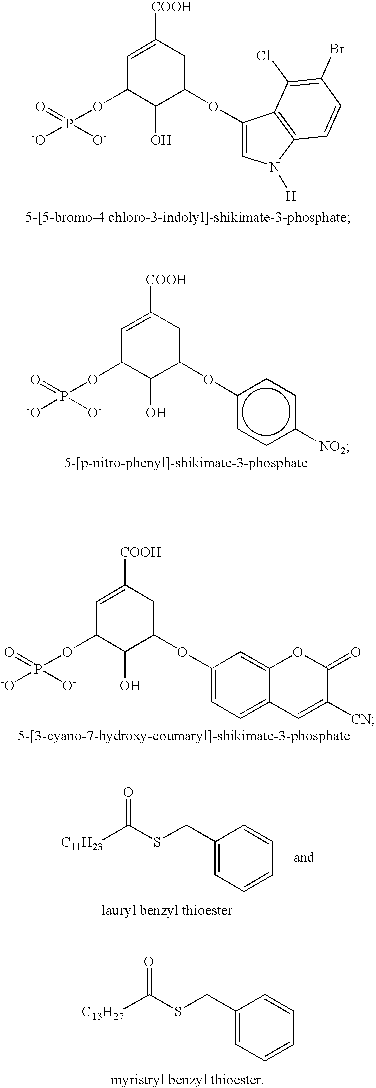

54. A compound selected from the group consisting of compounds according to the following structures:

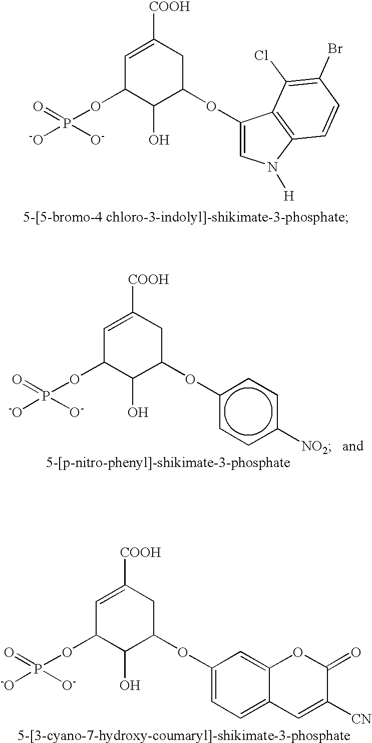

55. A compound selected from the group consisting of compounds according to the following structures:

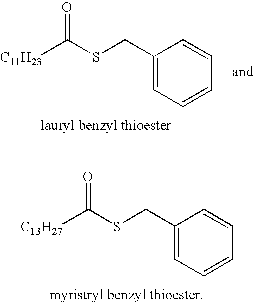

56. A kit useful in a method for detecting and/or separating a seed of a first plant from a seed of a second plant in a mixture thereof, wherein seeds of the first plant comprise a distinguishable marker, which marker is an enzyme, the kit comprising a detection agent and at least one compound selected from the group consisting of a surfactant, a selective inhibitor of the enzyme present in plant portions of said second plant, and combinations thereof.
